(19) United States
(12) Patent Application Publication
(10) Pub. No.: US 2004/0167385 A1
(43) Pub. Date: Aug. 26, 2004

(54) Catheter based sensing for intraluminal procedures

(76) Inventors: Robert F. Rioux, Ashland, MA (US); George Bourne, Southborough, MA (US)

Correspondence Address: Fay Kaplun & Marcin, LLP, Suite 702, 150 Broadway, New York, NY, 10038, US

(21) Appl. No.: 10/739,584

(22) Filed: Dec. 17, 2003

Related U.S. Application Data
(60) Provisional application No. 60/434,569, filed on Dec. 18, 2002.

(51) Int. Cl. A61B 5/04

(52) U.S. Cl. 600/373

(57) ABSTRACT

A system for embolotherapy comprises a catheter with a lumen extending therethrough from a proximal opening to a distal opening through which an embolic agent may be dispensed to a treatment site and at least one sensor coupled proximate to the distal end of the catheter to generate signals relating to a physiological condition in an area of the lumen adjacent to the sensor in combination with a controller receiving and processing the signals to generate data indicative of the physiological condition. A method of performing embolotherapy comprises inserting a catheter including at least one sensor mounted thereon into a blood vessel supplying a target tissue mass, the at least one sensor generating signals corresponding to a selected physiological condition in an area of the blood vessel adjacent thereto and processing signals generated by the at least one sensor prior to treatment of the target tissue mass to determine an initial state of the selected physiological condition in combination with treating the target tissue mass by dispensing an embolic agent from a distal end of the catheter, processing, after initiation of the treatment of the target tissue mass, the signals generated by the at least one sensor to determine a current state of the selected physiological condition and comparing the initial and current states to determine whether a desired change in the current physiological condition has been achieved.

### INCORPORATION BY REFERENCE

Applicants hereby expressly incorporate by reference the entire disclosure of U.S. Provisional Application Serial No. 60/434,569 filed Dec. 18, 2002.

### BACKGROUND OF THE INVENTION

Medical procedures for the treatment of diseased tissue masses such as tumors and fibroids use catheters to access the site of the diseased tissue mass and dispense therapeutic compounds directly to or near the tissue mass. While carrying out the procedures, physiological parameters associated with the diseased tissue may be monitored to assist in determining the degree of success of the treatment, and whether additional treatment is necessary.

Catheter based medical procedures can be monitored by physicians in a number of ways. Physicians may use a noninvasive imaging technique such as fluoroscopy, CAT scan or MRI to monitor a procedure in the body of a patient before, during and after the treatment. Noninvasive imaging provides information such as catheter placement, device placement, and to a lesser extent treatment site condition and treatment success. However, such monitoring has its limits. The machines used are expensive and require a highly trained operator. The images may not be of high quality because they are not based on in situ data, but rather are derived computationally by reconstructing indirect observations made using electrons, x-rays, etc. In addition, many imaging techniques require injection of a contrast agent into the patient which may cause additional problems.

Alternatively, catheter based devices or sensors may be used to directly monitor a limited number of parameters. For example, when performing electrophysiology procedures, sensing electrical activity within the heart can help diagnose aberrant electrical pathways in the tissue. These can then be treated immediately, often using the same catheter used for the sensing. After the treatment has been carried out, the catheter device may be used to evaluate the results, and determine if additional treatment is necessary.

### SUMMARY OF THE INVENTION

In one aspect, the present invention is directed to a system for embolotherapy comprising a catheter with a lumen extending therethrough from a proximal opening to a distal opening through which an embolic agent may be dispensed to a treatment site and at least one sensor coupled proximate to the distal end of the catheter to generate signals relating to a physiological condition in an area of the lumen adjacent to the sensor in combination with a controller receiving and processing the signals to generate data indicative of the physiological condition.

The present invention is further directed to a method of performing embolotherapy comprising inserting a catheter including at least one sensor mounted thereon into a blood vessel supplying a target tissue mass, the at least one sensor generating signals corresponding to a selected physiological condition in an area of the blood vessel adjacent thereto and processing signals generated by the at least one sensor prior to treatment of the target tissue mass to determine an initial state of the selected physiological condition in combination with treating the target tissue mass by dispensing an embolic agent from a distal end of the catheter, processing, after initiation of the treatment of the target tissue mass, the signals generated by the at least one sensor to determine a current state of the selected physiological condition and comparing the initial and current states to determine whether a desired change in the current physiological condition has been achieved.

### BRIEF DESCRIPTION OF THE DRAWINGS

FIG. 1 is a side elevation view showing an exemplary embodiment of a catheter with a sensor according to the invention;

FIG. 2 is a front elevation view of the embodiment shown in FIG. 1;

FIG. 3 is a side elevation view showing a second exemplary embodiment of a catheter with a sensor according to the invention;

FIG. 4 is a side elevation view showing a third exemplary embodiment of a catheter with a sensor according to the invention;

FIG. 5 is a side elevation view showing a fourth exemplary embodiment of a catheter with a sensor according to the invention;

FIG. 6 is a side elevation view showing a fifth exemplary embodiment of a catheter with a sensor according to the invention;

FIG. 7 is a side elevation view showing another exemplary embodiment of a catheter with a sensor according to the invention;

FIG. 8 is a side elevation schematic view showing an exemplary embodiment of a catheter with a sensor connected to a controller according to the invention;

FIG. 9 is a side elevation schematic view showing an exemplary embodiment of a catheter with a sensor connected to an electronic computer according to the invention;

FIG. 10 is a side elevation schematic view showing an exemplary embodiment of a catheter with a sensor connected to a display according to the invention;

FIG. 11 is a side elevation schematic view showing an exemplary embodiment of a catheter with multiple sensors according to the invention;

FIG. 12 is a side elevation schematic view showing an exemplary embodiment of a catheter located in a body lumen according to the invention;

FIG. 13 is a side elevation schematic view of the catheter shown in FIG. 12, during release of a medical compound according to the invention;

FIG. 14 is a side elevation schematic view of the catheter shown in FIG. 12, at a later time during release of the medical compound according to the invention; and

FIG. 15 is a side elevation schematic view showing a different exemplary embodiment of a catheter located in a body lumen according to the invention.

### DETAILED DESCRIPTION

The present invention may be further understood with reference to the following description and the appended drawings, wherein like elements are referred to with the same reference numerals. The invention is related to medical devices used to introduce therapeutic compounds into a body lumen and to monitor physiological conditions in the lumen. Specifically, the devices according to the invention may be used to perform embolotherapy procedures and to monitor data corresponding to the success of these procedures.

As indicated above, many medical procedures using catheters may benefit from a monitoring system integral with the catheter and which does not rely on external, indirect measurements of the parameters being monitored. Accordingly, the present invention provides a method and system by which the physician can directly monitor physiological parameters of interest at the site where the medical procedure takes place. In an exemplary embodiment, the medical procedure being carried out is an embolotherapy. In this procedure, en embolic agent is inserted through a catheter to a target portion of a blood vessel, to impede the flow of blood therethrough. As a result, the tissue receiving its supply of blood from the blood vessel no longer receives blood, and dies. This procedure may be used to treat tumors, fibroids, and other diseased tissue, by causing necrosis of the target tissue.

In the exemplary procedure, it is useful for the physician to know whether the supply of blood to the target tissue has been completely interrupted or reduced and, if reduced, to what extent. The method and system according to the present invention allows the calculation of a flow rate of blood through the blood vessel supplying the target tissue. The flow rate is calculated based on measurements of physiological parameters within the blood vessel from one or more sensors built into the catheter used to supply the embolic agent. For example, a flow sensor may be used for this purpose. Alternatively, the flow rate may be calculated based on pressure and/or temperature measurements by one or more sensors located at known positions on the catheter. Various other parameters may also be measured and processed as needed, depending on the specific details of the medical procedure.

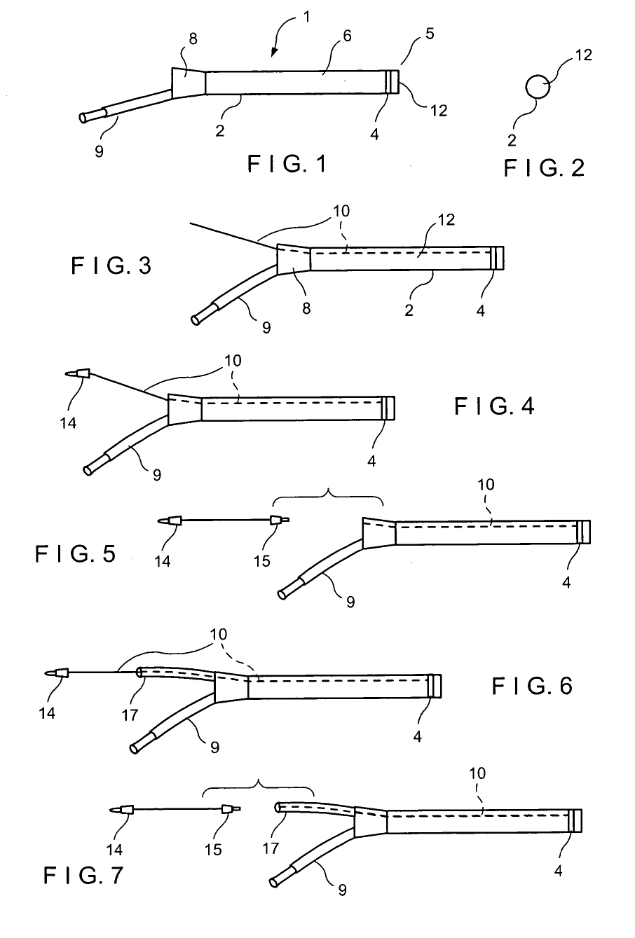

FIG. 1 illustrates an exemplary embodiment of a medical catheter system 1 according to the invention. The system comprises a catheter 2 used to deliver a therapeutic compound such as an embolic agent into the body of a patient. FIG. 1 illustrates an embodiment wherein the catheter 2 is a specialized catheter used to perform embolotherapy. The catheter 2 comprises a sensor 4 located on the outer surface thereof. In this example, the catheter 2 is a single lumen catheter having a body 6 constructed of known medical grade polymers, or combinations of known polymers as would be understood by those skilled in the art. As would be understood by those skilled in the art, these materials may include but are not limited to Pebax®, Flexima™, C-Flex®, nylon, polyethylene, PET, PTFE and LCP. The catheter body 6 is typically formed as an extrusion, pultrusion or molded catheter, but may be fabricated in any of the methods known in the art.

The exemplary catheter 2 may comprise any of the catheter constructs which are commonly used in the arts, including but not limited to reinforcements by braiding, ribbing, webbing, coils, layered metals, polymers or fabrics and the like. The catheter 2 may have variable stiffness along the length as a result of said constructs or through the use of joining materials, co-extruding materials, variable wall thickness, perforations and sheathing as would be understood by those skilled in the art. The catheter 2 may include tip constructions such as a soft atraumatic tip; preset curvatures; surface modifications and coatings such as hydrogels, Medi-Glide™, Hydropass®, and silicones. The catheter 2 may use any number of, styles of and modifications of internal lumens known in the art to achieve the medical treatment desired. A typical construction of the catheter 2 comprises an internal lumen that is round in cross-section but may alternatively have another geometry, such as a “D” shape, that is advantageous to the treatment or handling of the catheter 2. A multi-lumen catheter may be used to allow injection of more than one embolic agent, or of complimentary embolic agents to the treatment area. FIG. 2 illustrates a cross-section of a preferred embodiment of the catheter 2, having one internal lumen 12 of substantially circular cross section.

Some of the characteristics generally considered in the design and manufacture of medical catheters include providing a sufficiently small outer diameter to allow the catheter to easily pass through lumens to a target site along with sufficient lubricity, pushability, torqueability and non-kinking characteristics to enable the catheter to reach the target site without damaging surrounding tissue. It is also desirable to include an atraumatic tip to minimize injury to tissue adjacent to the path along which the catheter will travel and a construction which permits easy passage and delivery of a therapeutic compound to the target site.

In one exemplary embodiment, the catheter system 1 is adapted to deliver any known embolic agents to a target site within the body to perform an embolotherapy procedure. For example, representative but not limiting embolic agents used in conjunction with the catheter system 1 may include microspheres such as Contour® SE PVA microspheres manufactured by Boston Scientific Corp., Natick, Mass.; flakes; powders; liquids; gels; adhesives; polymers; particles; fibers; shavings or slivers; engineered geometries and the like. Furthermore, more than one embolic agent may be delivered at a time, particularly if the agents are complimentary such as agents which cooperate to pack and occlude a vessel, or which interact with each other (as is the case with a two part epoxy) to set the embolic agent. The embolic agent may include biological materials or agents, such as collagen, albumin, elastin or hyaluronic acid as would be understood by those skilled in the art.

In another exemplary embodiment, the embolic agent may also include therapeutic agents such as thrombotics, tissue growth factors, hormones, cytotoxins and cytostats. The embolic agent may be designed to be permanent, degradable or partially degradable, depending on the desired life span of the therapeutic intervention. The embolic agent may contain contrasting agents, particles or voids to aid imaging. The embolic agent may be coated for lubricity or agent delivery, and may be suspended in a carrier to aid injection, visualization, lubricity or occlusion. These examples are not meant to limit the possible types or schemes of embolic agent that may be components of the system of the instant invention, but are simply meant to illustrate several descriptive embodiments of the invention.

In another exemplary embodiment, the catheter 2 is a microcatheter such as the Renegade™ Hi-Flo catheter manufactured by Boston Scientific Corp. A typical microcatheter will have an outer diameter in the range of 2.2F-2.8F and may have any number of internal lumens—typically 1 to 3 lumens. However, microcatheters used in embolotherapy typically only have one internal lumen. When performing embolotherapy procedures it is important to use a catheter having an adequately sized internal lumen, to permit easy passage and delivery of the embolic agent(s). The inner diameter of the exemplary microcatheter may be in the range of about 0.016 in to about 0.030 in. As described above, the catheter 2 may be formed of any medical grade polymer known in the art or combination of polymers. For embolotherapy applications, the catheter 2 preferably is formed of PTFE, which may include reinforcement materials such as polymer fibers and metallic braiding.

The length of the exemplary catheter 2 may be in the range of about 105 cm to about 150 cm. Those skilled in the art will understand that for embolotherapy applications, the length is preferably about 150 cm. It will be apparent to those of skill in the art that the catheter 2 may be made of any reasonable length necessary to reach the target site within the body. As described in FIGS. 1 and 2, the catheter system 1 may include various additional components. A hub connector 8 may be used to connect to the catheter 2 an injection hub 9 adapted to receive an injection syringe containing an embolic agent or other similar source of the embolic agent. The hub 9 may comprise a conventional luer lock hub as is known in the art and may also include improvements to aid in embolotherapy, such as a tapered entry port as included in the Venturi™ tapered hub manufactured by Boston Scientific Corp. As will be described below, the hub 9 or the hub connector 8 may accommodate sensors, conductors, connectors, transmitters and other system components located on the catheter 2 as necessary to carry out various functions according to the present invention.

According to exemplary embodiments of the present invention, the catheter 2 may include at least one sensor 4 located on the elongated body 6, as illustrated in FIG. 1. The sensor 4 may be adapted to measure any desired physiological parameter, including but not limited to pressure, flow rate, temperature, fluid velocity, physical dimensions, vessel compliance, light reflectivity, spectral reflectivity, electrical activity, pH, saline content, gas content (such as content of oxygen or nitrogen), the presence of various chemicals (such as the presence of organic and inorganic compounds, drugs, proteins, fats, salts, sugars, DNA, cells, hormones, enzymes, tumor specific factors) and the like. The type of sensor is not meant to be limited by this list, since the sensor 4 may be any type of sensor which is useful in detecting and monitoring a physiological parameter in the body of a patient. In an exemplary embodiment the sensor detects and monitors a physiological parameter that is useful in performing an embolotherapy procedure. Such parameters include, but are not limited to blood pressure, blood or fluid flow rate, and blood temperature.

The sensor 4 of the exemplary embodiment is located on the outside of the elongated catheter body 6. Such location allows the sensor 4 to be in contact with the physiology being detected and monitored. Alternatively, the sensor 4 may be located on the inside of the catheter 2 or may be embedded within the wall of the catheter body 6. Placement of the sensor 4 on the inside of the catheter 2 or within a wall thereof may help to reduce the external profile of the catheter 2, without excessive interference with the ability of the sensor 4 to measure the physiological parameter(s) of interest. The sensor 4 may include independent means for securing to the catheter 2 or may require a mechanical attachment. For example, the sensor 4 may be mounted on a ring, base or clip which is mounted around the catheter 2. If the sensor 4 is located on the outside of the catheter 2, it may be secured to the catheter 2 by any means known in the art, including but not limited to swaging, insert molding, adhesives, melting, elasticity, shrinking, bumps or divots, melt holes, mechanical hooks, friction or interference or catheter aspects such as tackiness, compliance, surface roughness, bumps or divots and coatings, as would be understood by those skilled in the art.

In applications where the sensor 4 is embedded within the wall of catheter 2, the attachment may be carried out by melting the sensor 4 into the wall, by grooving the wall, by melting a tube layer over the wall, by embedding the sensor 4 during extrusion, or by mechanically forcing the sensor 4 into the wall. In cases where the sensor 4 is located inside the working lumen 12 of the catheter 2, it may be held in place using any of the attachment methods listed above. In addition, the sensor 4 may be held in place against the inner wall of the catheter working lumen by executing an expansion of the sensor 4 or of a base thereof. As will be appreciated by those of skill in the art, the exemplary methods of securing the sensor 4 to the catheter 2 are not limited to the described embodiments. Instead, any suitable method known in the art to secure the two components may be applied to this device.

In one exemplary embodiment shown in the drawings, the sensor 4 is located near the distal end of the catheter 2. When performing many medical applications using the catheter 2, including embolotherapy procedures, the location of the sensor 4 on the catheter is preferably near the treatment site in the body of a patient. During an embolotherapy procedure the distal end of the catheter 2 is closest to the treatment site, and the embolic agent is dispensed from the distal tip 5 of the catheter 2 for infusion into a lumen. As an example, the distal end of the catheter 2 may include the most distal 25% of the length of the catheter 2, and preferably the most distal portion extending between the distal tip and about 5% to about 10% of the length of the catheter 2. It will be understood by those skilled in the art that the exact location of the sensor(s) 4 along the catheter 2 may be dictated by the requirements of the medical procedure being performed.

In an exemplary embodiment, the sensor 4 is a thin film pressure sensor. The thin film pressure sensor 4 operates by reacting to surrounding fluid pressure with a change in electrical resistance. For example, the thin film sensor 4 may be designed to detect fluid pressures in the range of 0 to about 760 mmHg. In certain embodiments, the sensor 4 must be powered to operate. Electrical power may be delivered to the sensor 4 via conductors such as a conductor 10 extending the length of the catheter 2. The conductor 10 may consist of two independent conductors, one to power the sensor 4 and one to conduct the signal from the sensor 4 to a control system. As an alternative to these two conductors, the conductor 10 may consist of one conductor which is used both to transmit power and data between the sensor 4 and the proximal end of the catheter 2. For certain specialized applications, more than two conductors may be employed as required to operate the sensor(s) 4 successfully.

The conductor 10 may be embedded within the wall of the elongated catheter body 6, or may be located on internal surfaces of the working lumen 12 of the catheter 2. The catheter 2 is designed to minimize the impact of the conductor 10 on the functioning of the internal lumen 12, for example by using a small diameter conductor or a flat conductor which does not substantially reduce the internal diameter of the working lumen 12. The conductor 10 may be in the form of a wire, a foil, a conductive polymer or any of the power transmission means known in the art and may be co-extruded within the catheter 2 or included as part of a reinforcement of the catheter 2 (e.g., through inclusion in a reinforcing braiding thereof). Thus, the conductor 10 causes minimal impact on the mechanical characteristics of the catheter 2 by virtue of its minimal dimensions, material and integration into the catheter 2. In a different embodiment, the conductor 10 may comprise optic fibers used to convey data, control signals and power and to perform other functions.

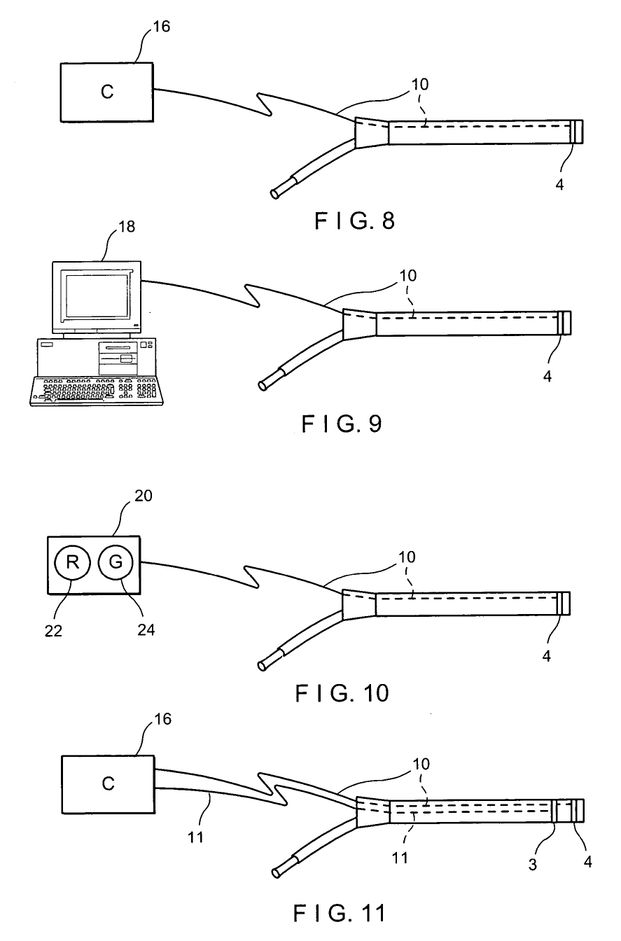

In the embodiments depicted in FIGS. 3-7, the conductor 10 is used to carry both power to and signals from the sensor 4. As illustrated in FIG. 4, one embodiment of the conductor 10 may include a connector 14 adapted to interface with a controller 16 or a signal readout system 20 (FIGS. 8-10). The connector 14 shown in FIGS. 3-7 may be any of the conventional electrical connectors known in the art, and is typically a bipolar jack used to both send power and read the signals from the sensor 4. Alternatively, the connector 14 may be substituted by a hard wire connection into the controller 16 or the signal readout system 20.

In embodiments where the conductor 10 is made integral to the catheter 2 (e.g., by being embedded or extruded into the catheter wall) the connector 14 may use an additional connector 15 to join the catheter 2 to an external control unit, as is illustrated in FIGS. 5 and 7. The connector 14 or a portion of the connector 14 may be made integral to the hub 9 or to the hub connector 8. Alternatively, the connector 14 may comprise a second hub 17, as illustrated in FIGS. 6 and 7. The hub 9 may be an injection hub for the injection of substances into the body, such as saline solution, therapeutic agents, or one or more embolic agents. The second hub 17 comprises the power and/or data connector 14, and may further comprise a lumen to allow infusion of another substance, such as one or more embolic agents into the same lumen 12 connected to the hub 9 or into a second lumen of the catheter 2.

In other exemplary embodiments, the conductor 10 may be a light conductor. The sensor 4 may be responsive to light reflection and may require to be powered. Batteries may be used to internally power the sensor 4, or another conductor may be provided for that use, as described above. Batteries may be located within the sensor 4 in the catheter 2, in the hub connector 8 or in the controller 16. Alternatively, the sensor 4 may be powered by the physiological environment in the patient's body which surrounds the sensor 4, or may be powered using a wireless technology such as by energy delivered through the body via microwaves. In the latter embodiment, the sensor 4 may comprise transmission electronics used to send signals though the body wirelessly.

The sensor 4 may be controlled by a controller 16, as illustrated in FIG. 8, which provides a source of power to the sensor 4 and which may, if necessary, include a system adapted to receive signals from the sensor 4, interpret these signals and display the data in a manner usable by the operating physician. In an exemplary embodiment illustrated in FIG. 9, the controller 16 comprises an electronic computer 18. According to this embodiment, the controller 16 may comprise a processor, a computer display and software, hardware or firmware adapted to operate the electronic computer 18 and the sensor 4. In an alternate embodiment illustrated in FIG. 10, the controller 16 comprises a two phase readout system 20 which preferably includes a component adapted to monitor a given signal level from the sensor 4 and additional electronic components adapted to process the signals from the sensor 4. The readout 20 may be adapted to indicate a change of state at a preset level in response to the signals received from the sensor 4, using a gauge or other indicator. For example, a minimum level, a maximum level or a preselected level of the signal may trigger a specific indication in the readout 20.

In one exemplary embodiment, the readout 20 comprises at least two indicator displays 22 and 24 as illustrated in FIG. 10, which may be color coded lights. The readout 20 may also comprise a power supply, hardware, firmware or software adapted to power the sensor 4 and to receive and interpret the signals from the sensor 4. The readout 20 may further comprise hardware, firmware or software adapted to set a preset level of the signal detected by the sensor 4, which may be hardwired or may be altered by the user. The readout 20 may further comprise hardware, firmware or software adapted to process and condition the signal from the sensor 4 and to select and power one or more indicators, such as the indicators 22 or 24. For example, the indicator 22 may be a red light display and the indicator 24 may be a green light display, which are turned on and off according to a selected convention to indicate the state of the physiological condition monitored by the sensor 4.

The readout 20 may include an internal power source, such as a battery, to power both the readout 20 and the sensor 4 and an on/off switch operable by the physician. The red light display 22, which may be set to indicate the normal operating condition, may activate at physiologic parameter levels detected by the sensor 4 that are below a preset parameter level. These physiological parameters may include but are not limited to pressure or flow through a blood vessel. When the sensor 4 is not inside the body of a patient and is exposed to normal ambient conditions, the red light display 22 may be activated. The green light display 24 may activate only above a detected preset physiological parameter level, which may be fixed in the device or may be altered by the user. The readout 20 may further comprise a means for the user to alter the preset physiological parameter level, including but not limited to a software instruction, a set screw or a dial.

In a different alternate embodiment of the system according to the invention, the catheter 2 comprises more than one the sensor 4. For example, an additional sensor 3 may augment the sensor 4 as illustrated in FIG. 4. The additional sensor 3 may be located on the outside of the catheter body 6, proximal to the sensor 4, or may be located at any point along the length of the catheter 2. In cases where the catheter 2 is utilized to perform embolotherapy procedures, the additional sensor 3 is located in the vicinity of the sensor 4, at a known distance from the sensor 4 which is a function of the physiological parameters being monitored.

The additional sensor 3 may be of the same type as the sensor 4, or may be a different type of sensor which measures a different physiological parameter. In cases where the additional sensor 3 is the same type of sensor as the sensor 4, the two sensors may have the same range and sensitivity, or may measure the parameter over different ranges and with different sensitivities. The additional sensor 3 may be redundant to the sensor 4, and may work independently of or in conjunction with sensor 4. In one embodiment, the sensors 3 and 4 are thin film pressure sensors having similar performance, and may be adapted to measure a difference in pressure along the length of the catheter 2 which is inserted in the body of a patient.

In one exemplary embodiment, the sensor 3 and the sensor 4 are thin film pressure sensors used to calculate blood flow rate along the length of the catheter 2. However, those skilled in the art will understand that the flow rate may be calculated based on data from a pair of temperature sensors positioned along the catheter 2 as described for the thin film pressure sensors. This arrangement is particularly useful when the catheter 2 is used in an embolotherapy procedure to introduce at least one embolic agent in the body of a patient. The sensors 3 and 4 cooperate to determine the fluid flow within a vessel feeding blood to a targeted diseased tissue site, such as a tumor or fibroid, before, during and after the embolotherapy procedure. In this embodiment, the sensors 3 and 4 are preferably located near the distal portion of the catheter 2, and preferably near the distal tip 5 thereof. The additional sensor 3 may be located approximately 20 mm closer to the proximal end of the catheter 2 than the sensor 4 and is more preferably located approximately 5 mm closer to the proximal end of the catheter 2 than is the sensor 4. The sensors 3 and 4 are preferably located as close to the distal tip 5 of the catheter 2 as is practical. For example, the sensor 4 may be preferably located within about 5 mm from the distal tip 5. However, the additional sensor 3 may be located at a greater distance from the sensor 4, for example, to detect fluid flow through a blood vessel side branch which is located proximal to the distal tip 5.

The descriptions and discussions above related to the sensor 4 apply equally to the additional sensor 3. A separate conductor 11 may be used to convey power and data between the additional sensor 3 and the proximal end of the catheter 2. However the conductor 10 may be used by both the sensors 3 and 4. In this embodiment, the controller 16 further comprises hardware, firmware or software to detect, interpret, process and condition the signal received from the sensor 3. The controller 16 may further comprise hardware, firmware or software to compute derived values from the signals received from both the sensors 3 and 4 and to provide a conditioned display signal based on the results of the computation. In an exemplary embodiment, the controller 16 may be adapted to interpret the fluid pressure reported by the sensors 3 and 4, calculate a pressure differential therebetween and to further calculate an actual, estimated or relative fluid flow rate. The result of the calculation may depend upon the other physiological parameters that are available, such as vessel diameter, flow temperature etc.

The catheter 2 may be used in any medical diagnosis or treatment which may benefit from the measurement of physiological parameters in the operative area before, during and after a procedure. The catheter 2 may be used in any location within the body of a patient. Examples of diagnostic and therapeutic procedures which may use the catheter according to embodiments of the invention include vascular embolotherapy, arteriosclerosis detection, vascular occlusion, cranial aneurysms, venous thrombosis, arterial and venous stenting procedures, cardiac monitoring, biliary strictures, arterial strictures; venous filtering; angioplasty; percutaneous fluid drainage, urethral drainage, central venous infusion and aspiration, drug delivery and the like. This list is by no means exhaustive and is not meant to be limited by the examples given.

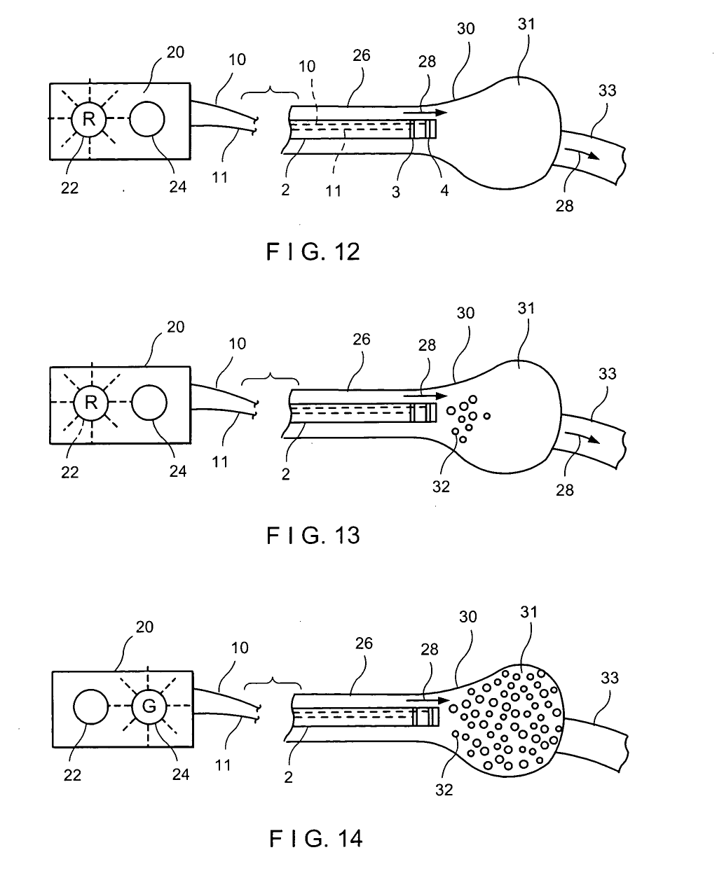

In one particular embodiment, the catheter 2 is an embolotherapy catheter used to deliver embolic compounds into the body of a patient to treat a diseased tissue 31 such as a tumor growth or a fibroid. FIGS. 12-14 illustrate an exemplary method for using an embodiment of an embolotherapy catheter according to the instant invention. In FIG. 12, the catheter 2 has been inserted into the body of a patient and advanced through a blood vessel 26 and up to a mouth 30 of a diseased tissue mass 31. The catheter 2 of the embodiment illustrated carries sensors 3 and 4, mounted on the outside surface thereof around a distal portion of the catheter 2. In this exemplary embodiment the catheter 2 is a microcatheter having an outer diameter that is smaller than the diameter of the surrounding blood vessel. As such, blood 28 is able to freely flow around the catheter 2, as is illustrated by the arrow, while the catheter 2 is being advanced in the blood vessel 26 and after the catheter 2 has reach a medical treatment site.

In the exemplary embodiment as described above, the sensors 3 and 4 are thin film pressure sensors used in conjunction to detect and calculate a blood flow rate around the catheter 2 based on a detected pressure differential between the two locations along the blood vessel 26. As the additional sensor 3 is proximal to the sensor 4, the additional sensor 3 will normally detect a higher blood pressure than the sensor 4. This pressure differential is a function of the fluid flow, among other parameters, and can be used to calculate an approximate fluid flow by using the following equation:

Q=ΔP/R

Where Q is the calculated fluid flow, ΔP is the pressure differential and R is the resistance of the vessel and catheter to fluid flow. R is calculated as a function of the vessel diameter and catheter diameter.

For the purposes of monitoring the progress of an embolotherapy procedure, the calculation of the actual fluid flow through a blood vessel is not crucial to the outcome, but it is a useful parameter. More pertinent to determining the success of the procedure is monitoring the relative flow of blood 28 before, during and after the embolic agent 32 has been delivered. To this end, once the distal end of the catheter 2 has reached the treatment site, the monitoring of fluid flow begins, and an initial state of the physiological condition being monitored is determined. If, for example, fluid flow is monitored, the computer 18 may display the initial flow rate, a current flow rate or a flow rate relative to the initial flow rate, as desired. The display of the relative flow rate may initially indicate any non-zero flow rate. If the fluid flow is monitored by the readout 20, the readout 20 may display a light indicating normal blood flow when the flow rate is within a predetermined range.

While performing an embolotherapy procedure, the catheter 2 is advanced within a blood vessel inside the body of a patient to a target site. The target site is typically the mouth 30 of a diseased target tissue mass 31 (e.g., a tumor or a fibroid) from which the blood vessel 26 feeds the target tissue 31 mass, as illustrated in FIG. 13. A blood vessel 33, which may be a vein, provides a return conduit for the blood 28 after leaving the target tissue mass 31. Once the catheter 2 has reached the target site, an initial reading of the measurement for the physiological parameter of interest is made by the controller. This initial reading of the signal is used to determine the baseline state of the physiological condition being monitored, such as the blood flow rate. However, an unexpected value of the baseline parameter may indicate problems with the apparatus. For example, a blockage in the vessel, a malfunctioning sensor, inaccurate placement of the catheter or other problems may be discovered early in the procedure.

Positioning of the catheter 2, the additional sensor 3 and the sensor 4 may be aided by noninvasive imaging techniques such as fluoroscopy. For example, if the sensors 3 and 4 are radiopaque, they will be readily visible with those techniques. Alternatively, the catheter 2 may be formed with a braid of strengthening material extending therein. For example, the Hi-Flo Microcatheter available from Boston Scientific Corp. of Natick, Mass. is suitable for this application and includes a platinum braid coextruded with the catheter. This platinum braid is radiopaque and is, therefore, visible using known non-invasive imaging techniques. Furthermore, if this braid of strengthening material is electrically conductive, as is the case with the platinum braid of the Hi-Flo Microcatheter, the braid may also provide the conductors 10 and 11 and any other conductors required. In addition, as would be understood by those skilled in the art, the braid may be designed with 2 or more parts electrically isolated from one another to provide independent paths for the various signals and/or power supply lines to the sensors 3 and 4.

Once the catheter 2 and the sensors 3 and 4 have been correctly positioned and the initial parameter measurement has been established, the treatment may be begun. The baseline may be interpreted, for example, as flow of blood or as a pressure differential and it may be displayed as such in a display unit of the readout 20, in an electronic computer 18, or using any other suitable interface. A baseline condition of the physiological parameter can thus be established, which is later compared to current measurements from the sensors 3 and 4 to determine whether a desired change in the current physiological condition has been achieved.

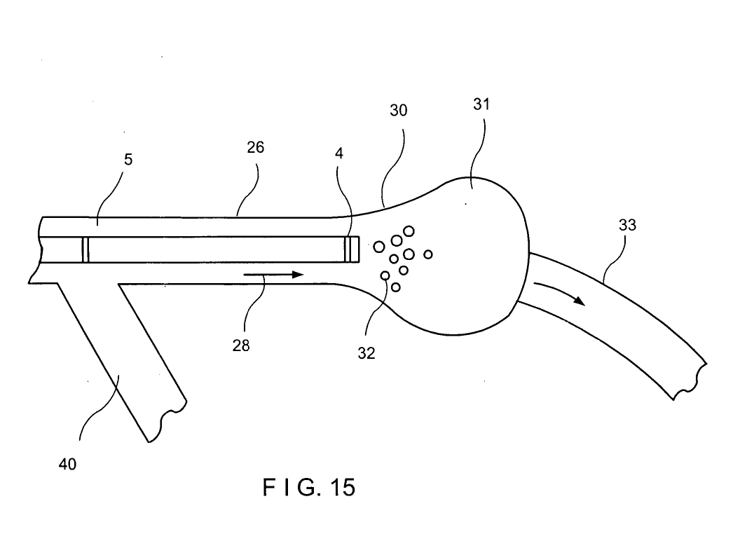

Carrying out the embolotherapy treatment according to the invention comprises infusing or introducing at least one embolic agent 32 into the mouth 30 of the blood vessel 26 supplying the target tissue mass 31, as illustrated in FIGS. 13, 14. The embolic agent 32 may be any one or combination of the embolic agents known in the art. In one exemplary embodiment, the embolic agents 32 comprise microspheres. While embolic agents 32 are being infused into the mouth 30, the sensors 3 and 4 detect and monitor the current condition of the physiological parameter, such as the blood pressure. The currently measured signals from the sensors 3 and 4 are sent via conductors 11 and 10, respectively, to the controller 16, to provide an up to date value of the current condition of the physiological parameter.

While the embolic agents 32 are being infused, the blood pressure within the blood vessel 26 may fluctuate. However, as long as blood 28 continues to flow past the sensors 3 and 4, at least a minimum pressure differential will be detected which may be interpreted by the controller 16 as a non zero flow rate (i.e., blood flow through the target tissue mass 31 has not yet been occluded). In one exemplary case, this condition causes the readout 20 to continue illuminating the red light display 22 indicating that blood continues to flow to the target tissue mass 31. The controller 16 may also be adapted to compare, on a running basis, a difference between the signals representing the baseline condition and the signals representing the current condition. The controller 16 may also be adapted to take some specified action, as will be described below, when the computed difference indicates that the current state has reached a selected condition of the physiological parameter, i.e. a specified blood flow rate.

The purpose of the embolotherapy procedure is to completely fill the mouth 30 of the target tissue mass 31 with embolic agents 32 to prevent blood flow therethrough. When the packing of the embolic agents 32 is satisfactory, blood flow through the mouth 30 will substantially stop and blood will no longer exit the target tissue mass 31 via the vessel 33. This reduction in blood flow will be detected by the sensors 3 and 4, for example, as a pressure differential substantially equal to zero. The readout 20 may indicate the no flow condition, for example, by activating the green light display 24. The physician thus receives a positive indication upon activation of the green light display 24 that the mouth 30 has been occluded, or more generally that a selected physiological condition has been reached, and may stop the infusion of the embolic agent 32 thereto. The process may be automated such that the software, hardware or firmware of the controller 16 or of the electronic processor 18 is adapted to terminate dispensing of the embolic agent 32 when the current state of the physiological condition measured by the sensors 3 and 4 reaches a selected value of the physiological condition. Of course, those skilled in the art will understand that a single display may change states with a first state indicating that the selected value of the physiological condition has not yet been reached and with a second state of the single display indicating that the selected value has been reached.

After the procedure has been completed, the catheter 2 may be left in position for a given amount of time to ensure that the embolic agents 32 do not loosen up or migrate, or that the flow of blood does not begin again for any reason. With the catheter 2 remaining in place to follow up on the procedure, if blood flow begins again the red light display 22 will activate to alert the physician that it is necessary to resume the infusion of the embolic agent 32. After the blood flow ceases again, the green light display 24 will be re-activated. This follow up process may be continued until the physician is confident that the flow of blood to the target tissue mass 31 has ceased permanently. The complete blood flow occlusion may be further verified by use of noninvasive imaging. Once satisfied that the procedure has been successful, the physician may retract the catheter 2 from the treatment site and complete the operation.

In a different embodiment of the invention, the additional sensor 3 may be located at a greater distance proximal from the sensor 4. In this case, the additional sensor 3 is placed sufficiently far from the sensor 4 to be able to detect the presence of a side branch blood vessel 40 in the vicinity of the distal tip 5, as is illustrated in FIG. 15. As the embolotherapy procedure takes place, the pressure in the vicinity of the sensor 4 will initially increase and then become steady, indicating a complete occlusion of the mouth 30 of the target tissue mass 31. However, due to the blood flow through the side branch 40, the additional sensor 3 will continue to detect a normal blood pressure fluctuation. If the additional sensor 3 later detects an increase in blood pressure and a lessening of the normal pulsation of the blood pressure, this condition may be interpreted to indicate a possible reflux of the embolic agent 32 out of the mouth 30 and a possible unwanted occlusion of the side branch 40.

The embolotherapy procedure may be monitored through the catheter system according to the invention by use of tandem thin film pressure sensors as described above, or by other types or numbers of sensors. In one additional embodiment, the catheter 2 may only have one sensor 4 adapted to measure a fluid pressure. As described above, the sensor 4 may be a thin film pressure sensor. With this configuration, a baseline reading may be established prior to injection of the embolic agent 32. As the embolic agent 32 is infused, the blood pressure measured by the single thin film pressure sensor 4 will increase until it stabilizes at a new level. Once the occlusion is complete, the blood pressure outside of the occlusion will no longer increase and the pressure average will plateau, although the normal blood pressure fluctuations will remain measurable. If catheter 2 is retained in place to monitor the occlusion, a change in the average pressure detected by the single sensor 4 may be interpreted, for example by controller 16, to indicate blood leakage into the mouth 30 of the target tissue mass 31. In response to this indication, the physician may infuse additional embolic agent 32 to stop the leakage.

In a different embodiment according to the present invention, the sensor 4 may be a temperature sensor, such as a thermistor or a thermocouple. As described above, when the catheter 2 is used in embolotherapy procedures it is useful to measure the flow rate of blood through a specified blood vessel. As blood flows by the sensor 4, it has a cooling or heating effect on the sensor 4 by convection heat transfer. For example, a thin wire or other easily cooled/heated structure may be heated to a temperature above that of the blood. When there is flow of blood around the thin wire, the flow will cool the wire, and result in a change of the wire's conductivity which may be measured and used to compute a blood flow rate. When the blood ceases to flow, the cooling effect will be reduced. The resulting change in temperature and conductivity of the sensor 4 can be extrapolated to indicate the successful occlusion of the mouth 30. As before, a change from a baseline initial measurement of pressure, temperature or flow velocity may be used to determine fluid stoppage.

In a further embodiments of the catheter system 1 of the instant invention, the device is provided as a kit to perform a specified medical procedure. The kit may comprise a catheter such as catheter 2 having at least one sensor such as the sensor 4. The entire catheter system 1 may be packaged as or included in the kit with other tools and devices used in the course of the medical procedure. In one embodiment, the catheter 2 is an embolotherapy catheter which is packaged with at least one embolic agent. Other items provided with an embolotherapy kit may include at least one syringe, guide wires, conductors 10 and readout gauge 20 along with a set of instructions for performing the methods described above. The controller 16, computer 18 and/or readout 20 may be provided with the kit, or may be provided separately. The description of items to be included in a kit or combinations of items to be included in a kit is not intended to be limited by the list provided above, but instead may include additional or fewer items.

The present invention has been described with reference to specific embodiments, and more specifically to a catheter with sensors used to measure flow in an embolotherapy procedure. However, other embodiments may be devised that are applicable to other medical devices and procedures, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive illustrative rather than restrictive sense.

What is claimed is:

1. A catheter comprising:
an elongated body having a distal end adapted for insertion into a body lumen and a proximal end opposite the distal end with a working lumen extending therethrough;
at least one sensor disposed on the elongated body generating a signal corresponding to a physiological condition; and
a transmission element adapted to convey the signal between the at least one sensor and an external controller.

2. The catheter according to claim 1, wherein the at least one sensor is a low profile sensor.

3. The catheter according to claim 2, wherein the at least one sensor is a thin film pressure sensor.

4. The catheter according to claim 2, wherein the at least one sensor is a temperature sensor.

5. The catheter according to claim 1, wherein the at least one sensor is disposed near the distal end of the elongated body.

6. The catheter according to claim 1, wherein the at least one sensor is responsive to one of a pressure, temperature, flow rate, flow velocity, reflectivity, electrical activity, chemical characteristics and chemical content of a body lumen within which the catheter is inserted.

7. The catheter according to claim 1, wherein the transmission element comprises at least one conductive element.

8. The catheter according to claim 1, wherein the transmission element comprises a wireless signal transmitter.

9. The catheter according to claim 1, wherein the at least one sensor comprises first and second sensors and wherein the signal comprises a first signal component from the first sensor and a second signal component from the second sensor.

10. The catheter according to claim 9, wherein the first and second sensors are pressure sensors.

11. The catheter according to claim 10, wherein the signal is adapted for processing to determine a flow rate of a fluid surrounding the first and second sensors.

12. The catheter according to claim 1, further comprising a connector of the transmission element disposed near the proximal end, the connector being adapted to receive an external wire.

13. A system for embolotherapy comprising:
a catheter with a lumen extending therethrough from a proximal opening to a distal opening through which an embolic agent may be dispensed to a treatment site;
at least one sensor coupled proximate to the distal end of the catheter to generate signals relating to a physiological condition in an area of the lumen adjacent to the sensor; and
a controller receiving and processing the signals to generate data indicative of the physiological condition.

14. The embolotherapy system according to claim 13, further comprising a display visually depicting the data indicative of the physiological condition.

15. The embolotherapy system according to claim 13, wherein the controller includes one of software, hardware and firmware calculating a blood flow rate based on the signals.

16. The embolotherapy system according to claim 15, wherein the at least one sensor includes a first pressure sensor.

17. The embolotherapy system according to claim 16, wherein the at least one sensor further comprises a second pressure sensor and wherein the blood flow rate is calculated based on a first pressure signal generated by the first pressure sensor and a second pressure signal generated by the second pressure sensor.

18. The embolotherapy system according to claim 15, wherein the at least one sensor includes a first temperature sensor.

19. The embolotherapy system according to claim 18, wherein the at least one sensor further comprises a second temperature sensor and wherein the blood flow rate is calculated based on a first temperature signal generated by the first pressure temperature sensor and a second temperature signal generated by the second temperature sensor.

20. The embolotherapy system according to claim 17, wherein the blood flow rate is calculated using the equation:
Q=ΔP/R
wherein Q is the blood flow rate, ΔP is a difference between the first and second pressure signals, and R is a resistance to fluid flow.

21. The embolotherapy system according to claim 13, wherein the controller determines an initial state of the physiological condition and a current state of the physiological condition.

22. The embolotherapy system according to claim 21, further comprising a flow control element controlling the dispensing of embolic agent to the lumen, wherein the controller operates the flow control element to terminate dispensing of the embolic agent when the current state reaches a selected condition.

23. The embolotherapy system according to claim 13, wherein the controller comprises an indicator providing a first indication when a selected physiological condition has not been reached and a second indication when the selected physiological condition has been reached.

24. The embolotherapy system according to claim 23, wherein the selected physiological condition is reached when a flow of blood past the at least one sensor is interrupted.

25. The embolotherapy system according to claim 17, wherein the first and second pressure sensors are disposed near the distal end of the catheter.

26. The embolotherapy system according to claim 19, wherein the first and second temperature sensors are disposed near the distal end of the catheter.

27. A method of performing embolotherapy comprising:
inserting a catheter including at least one sensor mounted thereon into a blood vessel supplying a target tissue mass, the at least one sensor generating signals corresponding to a selected physiological condition in an area of the blood vessel adjacent thereto;
processing signals generated by the at least one sensor prior to treatment of the target tissue mass to determine an initial state of the selected physiological condition;
treating the target tissue mass by dispensing an embolic agent from a distal end of the catheter;
processing, after initiation of the treatment of the target tissue mass, the signals generated by the at least one sensor to determine a current state of the selected physiological condition; and
comparing the initial and current states to determine whether a desired change in the current physiological condition has been achieved.

28. The method according to claim 27, further comprising placing a distal end of the catheter at a mouth of the blood vessel supplying the target tissue.

29. The method according to claim 27, wherein the selected physiological condition is a blood flow rate through the blood vessel.

30. The method according to claim 27, wherein the at least one sensor includes first and second pressure sensors, the method further comprising processing first and second pressure signals received from the first and second pressure sensors, respectively, to generate data corresponding to a blood flow rate in the blood vessel.

31. The method according to claim 27, displaying an indication at least one of the initial and current state of the selected physiological condition.

32. The method according to claim 27, further comprising terminating dispensation of the embolic agent when the desired change in the selected physiological condition has been achieved.
